(19) United States
(12) Patent Application Publication
(10) Pub. No.: US 2025/0090474 A1
(43) Pub. Date: Mar. 20, 2025

(54) BIOMIMETIC INTRAOCULAR DRUG DELIVERY SYSTEM FOR TREATMENT OF RETINOBLASTOMA AND METHODS THEREOF

(71) Applicant: THE UNIVERSITY OF HONG KONG, HONG KONG (CN)

(72) Inventors: Weiping WANG, Hong Kong (CN); Yichi ZHANG, Hong Kong (CN)

(73) Assignee: THE UNIVERSITY OF HONG KONG, HONG KONG (CN)

(21) Appl. No.: 18/889,021

(22) Filed: Sep. 18, 2024

Related U.S. Application Data
(60) Provisional application No. 63/583,620, filed on Sep. 19, 2023.

(51) Int. Cl. A61K 9/51; A61K 9/00; A61K 31/7048; A61P 27/02

(52) U.S. Cl. CPC A61K 9/5176; A61K 9/0048; A61K 9/5153; A61K 31/7048; A61P 27/02

(57) ABSTRACT

Provided is a drug delivery system comprising an ophthalmic composition for administration. The composition comprises nanoparticles comprising a core and an outer surface. The core comprises drugs and a carrier and the outer surface comprises a membrane derived from an ocular tumor cell. Also provided is a method of treating an ocular disease or condition using the nanoparticles. Also provided is a method of making the drug delivery system.

### RELATED APPLICATION

The present application claims priority to U.S. provisional application Ser. No. 63/583,620 filed Sep. 19, 2023, which is incorporated by reference in its entirety.

### 1. FIELD

Disclosed is an intraocular drug delivery system for treatment of eye disease and methods thereof.

### 2. BACKGROUND

Retinoblastoma is a kind of aggressive eye cancer usually happens in infancy and childhood. It has typical early-stage symptoms like leukocoria1. In recent years, there are around 9,000 new cases confirmed annually all over the world and the mortality even reaches 70% in some poor countries¹. In general, there are two main methods for treatment of retinoblastoma. One is enucleation of eyeballs. Even though it is an efficient method for prevention of spread of the tumor cells, such removal will lead to patients' loss of eyesight, especially for those with lesions at both sides. The other is systemic chemotherapy. This method is conservative but has severe side effects on off-target tissues and organs. Furthermore, the existence of blood-retina barrier (BRB) limits the delivery efficiency of the chemo drug². Consequently, intraocular chemotherapy has drawn much attention from researchers for treatment of retinoblastoma and other intraocular diseases³.

Biomimetic drug delivery system has been much investigated for its targeting effects and excellent biocompatibility through mimicking biological systems. For instance, pathogens like viruses can escape from recognition of immune systems. Furthermore, it can induce diseases through gene expression by inserting it into host cells. This feature makes viral structure suitable for being developed for gene delivery⁴. Cell membrane coating is another method for functionalization of nanoparticles.

For existence of some membrane surface proteins responsible for intercellular interaction, many cancer cells are reported to be able to adhere to each other, which can promote their growth and metastasis⁶.

Retinoschisin is a kind of extracellular cell surface protein mainly expressed and secreted by photoreceptor and bipolar cells of vertebrates⁸. It is reported that retinal structure is maintained by tight binding of retinoschisin among retinal cells⁹. Retinoschisin is largely expressed on retinoblastoma cells like WERI-Rb1 (RB1). However, there are no cancer cell membrane coated drug delivery systems reported for treatment of intraocular tumors like retinoblastoma. Thus, it is important to provide a drug delivery system and a treatment of intraocular tumors.

### 3. SUMMARY

Provided herein is an ophthalmic composition suitable for intraocular administration, the composition comprising a nanoparticle, the nanoparticle comprising:
(a) a core comprising one or more therapeutic agents and a carrier; and
(b) an outer surface surrounding the core, the outer surface comprising a membrane derived from an ocular tumor or ocular cancer.

In certain embodiments, the ocular cell is associated with an ocular disease or condition of an eye.

In certain embodiments, the ocular disease or condition is selected from the group consisting of ocular cancer, ocular tumor, macular degeneration, age-related macular degeneration (AMD), glaucoma, macular edema, uveitis, diabetic retinopathy, aphakia, blepharospasm, cataract, conjunctivitis, corneal disease, dry eye syndrome; an eyelid disease, a lacrimal disease, lacrimal duct obstruction, myopia, presbyopia, a pupil disorder, corneal neovascularization, a refractive disorder, strabismus, acute macular neuroretinopathy, Behcet's disease; choroidal neovascularization, histoplasmosis, multifocal choroiditis, proliferative vitreoretinopathy (PVR), retinal arterial occlusive disease, retinal detachment, sympathetic ophthalmia, Vogt Koyanagi-Harada (VKH) syndrome, a posterior ocular condition associated with an ocular laser treatment, a posterior ocular condition associated with photodynamic therapy, photocoagulation, radiation retinopathy, epiretinal membrane disorder, branch retinal vein occlusion, anterior ischemic optic neuropathy, non-retinopathy diabetic retinal dysfunction, and retinitis pigmentosa.

In certain embodiments, the membrane is or is derived from an ocular tumor or an ocular cancer.

In certain embodiments, the membrane is derived from an ocular cancer cell or tumor selected from the group consisting of retinoblastoma, medulloepithelioma, intraocular melanoma, iris melanoma, ciliary body melanoma, choroidal melanoma, uveal melanoma, eyelid cancer, intraocular lymphoma, orbital lymphoma, and a cancer that has metastasized to the eye.

In certain embodiments, the membrane is derived from retinoblastoma.

In certain embodiments, the therapeutic agent is a small molecule, a biomolecule, an enzyme, a protein, a polypeptide or a nucleic acid.

In certain embodiments, the therapeutic agent is an ophthalmic drug used to treat, prevent or diagnose an ocular disease, and wherein the therapeutic agent is selected from the group consisting of a chemotherapeutic agent, an anti-glaucoma agent, an anti-angiogenesis agent, an anti-infective agent, an anti-inflammatory agent, a growth factor, an immunosuppressant agent, an anti-allergic agent, and combinations thereof.

In certain embodiments, the therapeutic agent is a chemotherapeutic agent effective to treat an ocular cancer or an ocular tumor.

In certain embodiments, the chemotherapeutic agent is selected from the group consisting of etoposide, toposide, tebentafusp, mitomycin C, fluorouracil (5FU), methotrexate, cytarabine (Ara-C), thiotepa, chlorambucil, dacarbazine, temozolomide, teniposide, verapamil, vincristine, calcitriol, melphalan, cyclosporin, carboplatin, cisplatin, topotecan and Nutlin-3.

In one embodiment, the membrane comprises retinoschisin.

In certain embodiments, the carrier comprises a polymer, a lipid, a phospholipid, a dendrimer or a metal nanoparticle.

In certain embodiments, the carrier in the nanoparticle core comprises a biodegradable or biocompatible polymer.

In certain embodiments, the biodegradable or biocompatible polymer is selected from the group consisting of poly(D,L-lactide-co-glycolide) (PLGA), poly(L-lactide-co-glycolide), poly(D-lactide-co-glycolide), poly-(D-lactic acid) (PDLA), poly-(L-lactic acid) (PLLA), poly(D,L-lactic acid) (PLA), poly(glycolic acid) (PGA), poly(L-lactide-co-caprolactone), poly(D-lactide-co-caprolactone), poly(D,L-lactide-co-caprolactone) (PCL), polyvinyl alcohol (PVA), poly(ethylene glycol) (PEG), polylysine, poly(L-lactide-co-trimethylene carbonate), poly(D-lactide-co-trimethylene carbonate), poly(D,L-lactide-co-trimethylene carbonate), poly(trimethylene carbonate-co-ε-caprolactone), poly(caprolactone-co-glycolide), polyorthoesters, polyanhydrides, polyesters, polyamides, polyesteramides, polycarbonates, polyethylene, polypropylene, polygalactic acid, polyacrylamide, polystyrene, polyurethane, silicone, hydroxypropylmethylcellulose (HPMC), hydroxypropylcellulose (HPC), polyglycolic acid-polyvinyl alcohol copolymers, polycaprolactone-polyethylene glycol copolymers, collagen, croscarmellose collagen, hyaluronic acid, elastin, polyhydroxybutyrate, polyalkaneanhydrides, gelatin, cellulose, oxidized cellulose, polyphosphazene, poly(sebacic acid), poly(ricenolic acid), poly(fumaric acid), chitin, chitosan, polyvinylpyrrolidone (PVP), synthetic cellulose esters, polyacrylic acids, polybutyric acid; triblock copolymers (e.g., PLGA-PEG-PLGA, PEG-PLGA-PEG), poly(N-isopropylacrylamide, poly(ethylene oxide)-poly(propylene oxide)-poly(ethylene oxide) tri-block copolymers (PEO-PPO-PEO), poly valeric acid, poly(valerolactone), polyhydroxyalkylcellulose; siloxane, polysiloxane; dimethylsiloxane/-methylvinylsiloxane copolymer; poly(dimethylsiloxane/-methylvinylsiloxane/-methylhydrogensiloxane) dimethylvinyl or trimethyl copolymer, polypeptides, poly(amino acids), poly(dioxanones), poly(alkylene alkylates), hydrophobic polyethers, polyetheresters, polyacetals, polycyanoacrylates, polyacrylates, polymethylmethacrylates, polysiloxanes, poly(oxyethylene)/poly(oxypropylene) copolymers, polyketals, polyphosphates, polyhydroxyvalerates, polyalkylene oxalates, polyalkylene succinates, poly(maleic acid), as well as copolymers thereof or derivatives thereof including oxidized, reduced, mono substituted, disubstituted, multi-substituted derivatives, or any combinations thereof.

In certain embodiments, the carrier in the nanoparticle core comprises a biodegradable polymer selected from the group consisting of poly(D,L-lactide-co-glycolide) (PLGA), poly(L-lactide-co-glycolide), poly(D-lactide-co-glycolide), poly-(D-lactic acid) (PDLA), poly-(L-lactic acid) (PLLA), poly(D,L-lactic acid) (PLA), and poly(glycolic acid) (PGA). In certain embodiments, the weight ratio of the nanoparticle core to the membrane is about 10:1 to about 1:10.

In certain embodiments, the weight ratio of the nanoparticle core to the membrane is about 1:1.

In certain embodiments, the composition is in a form suitable for subcutaneous, subconjunctival, sub-tenon, intracameral, intravitreal or retrobulbar administration into an eye of a subject.

In certain embodiments, the ophthalmic composition is formulated as an intraocular injection or an ophthalmic implant.

In certain embodiments, the one or more therapeutic agents is selected from the group consisting of a chemotherapeutic agent, an anti-glaucoma agent, an anti-angiogenesis agent, an anti-infective agent, an anti-inflammatory agent, a growth factor, an immunosuppressant agent, an anti-allergic agent, and combinations thereof.

Provided herein is a method of treating an ocular disease or condition of an eye, comprising the step of intraocularly administering to a subject in need thereof an ophthalmic composition comprising a nanoparticle, the nanoparticle comprising:
(a) a core comprising one or more therapeutic agents and a carrier; and
(b) an outer surface surrounding the core, the outer surface comprising a membrane derived from an ocular tumor or ocular cancer.

In certain embodiments, the membrane is derived from a tissue associated with the ocular disease or condition.

In certain embodiments, the membrane is derived from retinoblastoma.

In certain embodiments, the ocular disease or condition is selected from the group consisting of ocular cancer, ocular tumor, macular degeneration, age-related macular degeneration (AMD), glaucoma, macular edema, uveitis, diabetic retinopathy, aphakia, blepharospasm, cataract, conjunctivitis, corneal disease, dry eye syndrome; an eyelid disease, a lacrimal disease, lacrimal duct obstruction, myopia, presbyopia, a pupil disorder, corneal neovascularization, a refractive disorder, strabismus, acute macular neuroretinopathy, Behcet's disease; choroidal neovascularization, histoplasmosis, multifocal choroiditis, proliferative vitreoretinopathy (PVR), retinal arterial occlusive disease, retinal detachment, sympathetic ophthalmia, Vogt Koyanagi-Harada (VKH) syndrome, a posterior ocular condition associated with an ocular laser treatment, a posterior ocular condition associated with photodynamic therapy, photocoagulation, radiation retinopathy, epiretinal membrane disorder, branch retinal vein occlusion, anterior ischemic optic neuropathy, non-retinopathy diabetic retinal dysfunction, and retinitis pigmentosa.

In certain embodiments, the uptake of the nanoparticle into an ocular cell is increased relative to a composition lacking the membrane.

In one embodiment, the membrane targets the ophthalmic composition to an ocular cell of the subject.

In one embodiment, the membrane is or is derived from an ocular tumor or an ocular cancer.

In certain embodiments, the membrane is derived from an ocular cancer cell selected from the group consisting of retinoblastoma, medulloepithelioma, ocular melanoma, intraocular melanoma, iris melanoma, ciliary body melanoma, choroidal melanoma, uveal melanoma, eyelid cancer, ocular lymphoma, intraocular lymphoma, orbital lymphoma, or a cancer that has metastasized to the eye.

In one embodiment, the membrane comprises retinoschisin.

In certain embodiments, the therapeutic agent is a small molecule, a biomolecule, an enzyme, a protein, a polypeptide or a nucleic acid.

In certain embodiments, the therapeutic agent is an ophthalmic drug used to treat, prevent or diagnose an ocular disease, and wherein the therapeutic agent is selected from the group consisting of a chemotherapeutic agent, an anti-glaucoma agent, an anti-angiogenesis agent, an anti-infective agent, an anti-inflammatory agent, a growth factor, an immunosuppressant agent, an anti-allergic agent, and combinations thereof.

In certain embodiments, the therapeutic agent is a chemotherapeutic agent effective to treat an ocular cancer or an ocular tumor.

In certain embodiments, the chemotherapeutic agent is selected from the group consisting of etoposide, toposide, tebentafusp, mitomycin C, fluorouracil (5FU), methotrexate, cytarabine (Ara-C), thiotepa, chlorambucil, dacarbazine, temozolomide, teniposide, verapamil, vincristine, calcitriol, melphalan, cyclosporin, carboplatin, cisplatin, topotecan and Nutlin-3.

In certain embodiments, the nanoparticle core comprises a biodegradable or biocompatible polymer.

In certain embodiments, the biodegradable or biocompatible polymer is selected from the group consisting of poly(D,L-lactide-co-glycolide) (PLGA), poly(L-lactide-co-glycolide), poly(D-lactide-co-glycolide), poly-(D-lactic acid) (PDLA), poly-(L-lactic acid) (PLLA), poly(D,L-lactic acid) (PLA), poly(glycolic acid) (PGA), poly(L-lactide-co-caprolactone), poly(D-lactide-co-caprolactone), poly(D,L-lactide-co-caprolactone) (PCL), polyvinyl alcohol (PVA), poly(ethylene glycol) (PEG), polylysine, poly(L-lactide-co-trimethylene carbonate), poly(D-lactide-co-trimethylene carbonate), poly(D,L-lactide-co-trimethylene carbonate), poly(trimethylene carbonate-co-ε-caprolactone), poly(caprolactone-co-glycolide), polyorthoesters, polyanhydrides, polyesters, polyamides, polyesteramides, polycarbonates, polyethylene, polypropylene, polygalactic acid, polyacrylamide, polystyrene, polyurethane, silicone, hydroxypropylmethylcellulose (HPMC), hydroxypropylcellulose (HPC), polyglycolic acid-polyvinyl alcohol copolymers, polycaprolactone-polyethylene glycol copolymers, collagen, croscarmellose collagen, hyaluronic acid, elastin, polyhydroxybutyrate, polyalkaneanhydrides, gelatin, cellulose, oxidized cellulose, polyphosphazene, poly(sebacic acid), poly(ricenolic acid), poly(fumaric acid), chitin, chitosan, polyvinylpyrrolidone (PVP), synthetic cellulose esters, polyacrylic acids, polybutyric acid; triblock copolymers (e.g., PLGA-PEG-PLGA, PEG-PLGA-PEG), poly(N-isopropylacrylamide, poly(ethylene oxide)-poly(propylene oxide)-poly(ethylene oxide) tri-block copolymers (PEO-PPO-PEO), poly valeric acid, poly(valerolactone), polyhydroxyalkylcellulose; siloxane, polysiloxane; dimethylsiloxane/-methylvinylsiloxane copolymer; poly(dimethylsiloxane/-methylvinylsiloxane/-methylhydrogensiloxane) dimethylvinyl or trimethyl copolymer, polypeptides, poly(amino acids), poly(dioxanones), poly(alkylene alkylates), hydrophobic polyethers, polyetheresters, polyacetals, polycyanoacrylates, polyacrylates, polymethylmethacrylates, polysiloxanes, poly(oxyethylene)/poly(oxypropylene) copolymers, polyketals, polyphosphates, polyhydroxyvalerates, polyalkylene oxalates, polyalkylene succinates, poly(maleic acid), as well as copolymers thereof or derivatives thereof including oxidized, reduced, mono substituted, disubstituted, multi-substituted derivatives, or any combinations thereof.

In certain embodiments, the nanoparticle core comprises a biodegradable polymer selected from the group consisting of poly(D,L-lactide-co-glycolide) (PLGA), poly(L-lactide-co-glycolide), poly(D-lactide-co-glycolide), poly-(D-lactic acid) (PDLA), poly-(L-lactic acid) (PLLA), poly(D,L-lactic acid) (PLA), and poly(glycolic acid) (PGA).

In certain embodiments, the weight ratio of the nanoparticle core to the membrane is about 10:1 to about 1:10.

In certain embodiments, the weight ratio of the nanoparticle core to the membrane is about 1:1.

In certain embodiments, the ophthalmic composition is in a form suitable for intraocular administration.

In certain embodiments, the ophthalmic composition is in a form suitable for subcutaneous, subconjunctival, sub-tenon, intracameral, intravitreal or retrobulbar administration, or in a form suitable for intravitreal administration.

In certain embodiments, the ophthalmic composition is formulated as an intraocular injection or an ophthalmic implant.

In certain embodiments, the method further comprising the step of administering one or more additional therapeutic agents.

In certain embodiments, the one or more therapeutic agents is selected from the group consisting of a chemotherapeutic agent, an anti-glaucoma agent, an anti-angiogenesis agent, an anti-infective agent, an anti-inflammatory agent, a growth factor, an immunosuppressant agent, an anti-allergic agent, and combinations thereof.

Provided herein is a method of making the drug delivery system comprising the steps of: (i) extracting an ocular tumor cell membrane to form an extract; (ii) loading a nanoparticle with one or more therapeutic agents and a carrier to form a core; (iii) combining the extract and the core to form the drug delivery system.

In certain embodiments, the therapeutic agent is in a range of about 0.01-0.04 mg/kg.

In certain embodiments, the therapeutic agent is in the range of about 0.0269 mg/kg.

In certain embodiments, the therapeutic agent is Etoposide.

### 4. BRIEF DESCRIPTION OF THE FIGURES

The patent or application file contains at least one drawing executed in color. Copies of this patent or application publication with color drawings will be provided by the Office upon request and payment of the necessary fee.

FIGS. 1A-1B (FIG. 1A) Scheme of membrane coating on PLGA@Drug nanoparticles (PLGA@Drug NPs). (FIG. 1B) Targeting effect of WERI-Rb1 membrane-coated PLGA@Drug nanoparticles (RB1-PLGA@Drug NPs) after intraocular injection.

FIGS. 2A-2E. show characterization and optimization of RB1 membrane coating on PLGA based nanoparticles. (FIG. 2A) TEM images of bare PLGA NPs, RB1 membrane vesicles, and RB1-PLGA NPs. (Scale bar: 200 nm) (FIG. 2B) Membrane protein analysis of the RB1-PLGA NPs. (FIG. 2C) Average size of WERI-Rb1 membrane-coated PLGA@Etoposide nanoparticles (RPE NPs) in water, PBS buffer (1×), and PBS buffer (1×) after 14 days under different weight ratio of RB1 membrane over PLGA@Etoposide nanoparticles (PE NPs) core. (FIG. 2D) Size distribution change of PE NPs before and after RB1 membrane coating. (FIG. 2E) Surface charge of PE NPs, RB1 membrane vesicles, and RPE NPs.

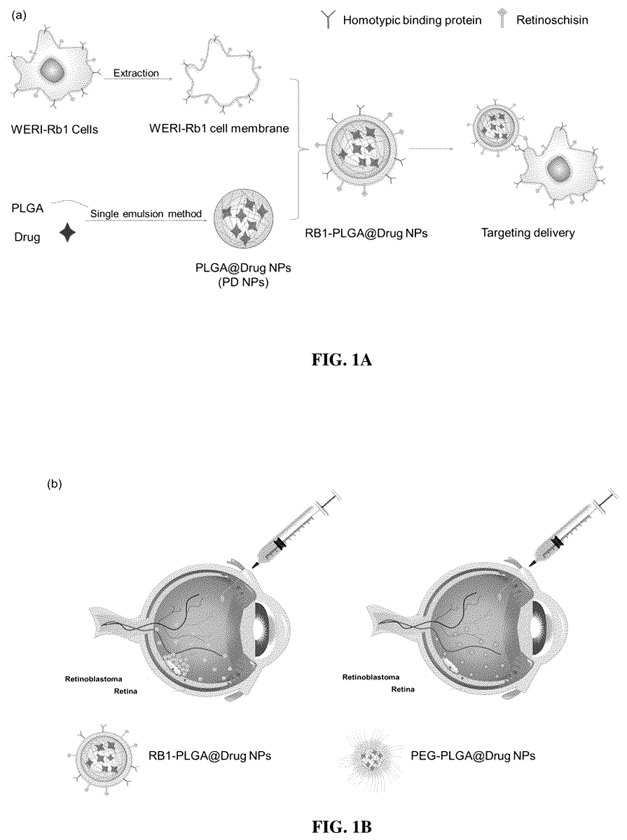

FIGS. 1A-3D show targeting effect of WERI-Rb1 membrane-coated PLGA@DiD nanoparticles (RPDiD NPs) on WERI-Rb1 cells. (FIG. 3A) Quantification of WERI-Rb1 cells' uptake of mPEG-PLGA@DiD nanoparticles (PPDiD NPs) and RPDID NPs. (FIG. 3B) Flow cytometry result of the uptake test. (FIG. 3C) Confocal images of the uptake (Scale bar: 10 μm). (FIG. 3D) Quantification of uptake of RPDID NPs by WERI-Rb1, 4T1 and KYSE450.

FIGS. 4A-4B. (FIG. 4A) Enhanced cytotoxicity of RPE NPs on WERI-Rb1 cells. (FIG. 4B) Stability test of the RPE NPs in PBS buffer (1×) in 25° C.

FIGS. 5A-5D. (FIG. 5A) Establishment of orthotopic tumor-bearing mouse model. (FIG. 5B) Representative images of mice intraocular fluorescence at different timepoints after intravitreal injection. (FIG. 5C) Quantitative analysis of retention rate of RPDiR NPs at different timepoints after intravitreal injection. (FIG. 5D) Intraocular distribution of DiR dye 72 h after injection. RPDiR NPs: WERI-Rb-1 cell membrane-coated PLGA@DiR nanoparticles. PPDiR NPs: mPEG-PLGA@DiR nanoparticles.

FIGS. 6A-6F. (FIG. 6A) Scheme of in vivo antitumour efficacy experiment. (FIG. 6B) Tumor progression profile. (FIG. 6C) Body weight change of the mice. (FIG. 6D) Representative IVIS tumor luminescence images. (FIG. 6E) Representative photos of tumor-inoculated eyeballs of mice after different treatments. (FIG. 6F) Representative H&E staining images of tumor-inoculated eyeballs of mice after different treatments. RPE NPs: WERI-Rb-1 cell membrane-coated PLGA@Etoposide nanoparticles. PPE NPs: mPEG-PLGA@Etoposide nanoparticles.

### 4.1 DEFINITIONS

The term “nanoparticle” as used herein, refers generally to a particle with at least one dimension at the nanoscale, particularly with all three dimensions at the nanoscale (1-200 nm). In this description, the nanoparticles are in the range 20 nm to 200 nm, including 20, 30, 40, 50, 60, 70, 80, 90, 100, 150 and 200. More particularly in the range from 50 to 150 nm. As regards the shape of the nanoparticles described herein, there are included spheres and polyhedral. In a particular embodiment the nanoparticle is spherical.

The term “size” refers to a characteristic physical dimension. For example, in the case of a nanoparticle that is substantially spherical, the size of the nanoparticle corresponds to the diameter of the nanoparticle. When referring to a set of nanoparticles as being of a particular size, it is contemplated that the set of nanoparticles can have a distribution of sizes around the specified size. Thus, as used herein, a size of a set of nanoparticles can refer to a mode of a distribution of sizes, such as a peak size of the distribution of sizes. In addition, when not perfectly spherical, the diameter is the equivalent diameter of the spherical body including the object.

As used herein, an “effective amount” of an agent is an amount sufficient to accomplish a specified task or function desired of the agent. A “therapeutically effective amount” of a composition, drug, or agent refers to a non-toxic, but sufficient amount of the composition, drug, or agent, to achieve therapeutic results in treating or preventing a condition for which the composition, drug, or agent is known or intended to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician, veterinarian, or other qualified medical personnel using evaluations known in the art, it is recognized that individual variation and response to treatments may make the achievement of therapeutic effects a somewhat subjective decision. The determination of an effective amount or therapeutically effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986). As used herein, a “dosing regimen” or “regimen” such as “treatment dosing regimen,” or a “prophylactic dosing regimen” refers to how, when, how much, and for how long a dose of an active agent or composition can or should be administered to a subject in order to achieve an intended treatment or effect.

As used herein, the terms “treat,” “treatment,” or “treating” refers to administration of a therapeutic agent to subjects who are either asymptomatic or symptomatic. In other words, “treat,” “treatment,” or “treating” can be to reduce, ameliorate or eliminate symptoms associated with a condition present in a subject, or can be prophylactic, (i.e. to prevent or reduce the occurrence of the symptoms in a subject). Such prophylactic treatment can also be referred to as prevention of the condition.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients. Compositions can take nearly any physical state, including solid, liquid (e.g. solution), or gas. Furthermore, the term “dosage form” can include one or more formulation(s) or composition(s) provided in a format for administration to a subject. For example, an injectable dosage form would be a formulation or composition prepared in a manner that is suitable for administration via injection.

As used herein, a “subject” refers to an animal. In one aspect the animal may be a mammal. In another aspect, the mammal may be a human. The term “pharmaceutically acceptable” as used herein pertains to compounds, materials, compositions, and/or dosage forms which are, within the scope of sound medical and veterinary judgment, suitable for use in contact with the tissues of a subject (e.g. human or any other animal) without significant toxicity, irritation, allergic response, or other problem or complication, commensurate with a reasonable benefit/risk ratio. Each carrier, excipient, etc., must also be “acceptable” in the sense of being compatible with the other ingredients of the pharmaceutical composition. It must also be suitable for use in contact with the tissue or organ of humans and animals without excessive toxicity, irritation, allergic response, immunogenicity or other problems or complications commensurate with a reasonable benefit/risk ratio. Suitable carriers, excipients, etc. can be found in standard pharmaceutical texts, and include, as a way of example preservatives, agglutinants, humectants, emollients, tonicity agents to adjust osmolality, chelating agents, and antioxidants.

### 5. DETAILED DESCRIPTION

Provided herein is a drug delivery system comprising a nanoparticle comprising: (a) a core comprising one or more therapeutic agents and a carrier; and (b) an outer surface surrounding the core, wherein the outer surface comprising a membrane derived from an ocular tumor or ocular cancer.

In one embodiment, the coated nanoparticles comprise high anchoring ability on ocular surface. In one embodiment, the composition provides long retention of the nanoparticles on ocular surface. In certain embodiments, the membrane has affinity towards an ocular tumor or ocular cancer.

In one embodiment, provided herein is a drug delivery system via intraocular injection. In one embodiment, provided herein is an eyedrop. In one embodiment, provided herein is a composition comprising human retinoblastoma cell membrane coated and drug-loaded nanoparticles.

### 5.1 Nanoparticles

In certain embodiments, the nanoparticle comprising: (a) a core comprising one or more therapeutic agents and a carrier; and (b) an outer surface surrounding the core, the outer surface comprising a membrane derived from an ocular tumor or ocular cancer.

In certain embodiments, the core comprises a carrier. In certain embodiments, the carrier comprises one or more polymer.

In certain embodiments, the nanoparticles comprises Poly(lactic-co-glycolic acid) (PLGA) polymer. PLGA can be polymerized with different ratios of lactic acid and glycolic acid (PLA: PGA) to create different forms of PLGA, which can affect the polymer's hydrophobicity, crystallinity, mechanical properties, size, and biodegradation rate. For example, PLGA 50:50 used in nanotechnology, is made up of 50% lactate and 50% glycolate. PLGA with a 75:25 or 50:50 molar ratio of lactic acid to glycolic acid exists in an amorphous state.

PLGA nanoparticles can be used to deliver a wide range of materials, including small molecules, macromolecules, peptides, and proteins. They can offer sustained release and targeted drug delivery and can be surface engineered. For example, PLGA nanoparticles can be conjugated with aptamers to target specific cells, such as breast adenocarcinoma cells or prostate cancer cells.

Exemplary biodegradable materials that can be used in the present disclosure include, but not limited to, PLGA (poly(lactic-co-glycolic acid)), PLA (polylactic acid), PLC (polylactide-caprolactone copolymer), PGA (polyglycolic acid), hyaluronic acid, collagen, SAIB (sucrose acetate isobutyrate), poly(orthoesters), PEG (polyethylene glycol), alginate, PCL (polycaprolactone), PCE (polycaprolactone-polyethylene glycol), PCEL (polycaprolactone-polyethylene glycol-polylactide) and PHB (poly-β-hydroxybutyrate).

Nanotechnology-based ocular drug delivery platforms could increase drug bioavailability to the eye. In particular, because a nanoparticle formulation will tend to accumulate in the conjunctival cul-de-sac, the contact time of the nanoparticle drug is considerably longer than comparable ophthalmic solutions. This increased contact time may give a longer duration of action.

The term “nanoparticle” encompasses particles, nanospheres, nanocapsules, liposomes, polymeric micelles, quantum dots, dendrimers, solid lipid nanoparticles, etc. Examples of nanoparticle formulations that could be used for ocular drug delivery are described in Deepak Thassu & Gerald Chader (eds.), Ocular Drug Delivery Systems: Barriers and Application of Nanoparticulate Systems (2013) CRC Press and Kewal K. Jai n, “Nanocarriers for Ocular Drug Delivery” in The Handbook of Nanomedicine (2008) Humana Press.

The nanoparticles may be made of any suitable material, including biocompatible polymers or biologic materials. Examples of such materials include chitosan, a polycarboxylic acid such as polyacrylic acid, hyaluronic acid esters, polyitaconic acid, poly(butyl) cyanoacrylate, poly-ε-caprolactone, poly(isobutyl) caprolactone, poly(lactic/glycolic) acid, or poly(lactic acid), poly(ethylene glycol)-block-poly(L-lysine). In certain embodiments, copolymers of ethyl acrylate, methyl methacrylate, and a low content of methacrylic acid ester with quaternary ammonium groups may be used.

In some embodiments, the nanoparticles comprise polymers that are both biocompatible and biodegradable, such as polylactic acid, polyglycolic acid, poly(lactic/glycolic) acid, poly(caprolactone), polyhydroxobutyrate, chitosan, hyaluronic acid, poly(2-hydroxyethyl-methacrylate), and poly(ethylene glycol). In some cases, the nanoparticles comprise synthetically-made polymers that are both biocompatible and biodegradable.

The nanoparticles may have any suitable size less than 1 μm. In some embodiments, the nanoparticles have an average size diameter in the range of 100-700 nm. The resulting nanoparticle formulation may be in any suitable liquid form, including suspension, emulsion, gel, sol, liquid foam, etc.

In certain embodiments, nanoparticles can include a nanoemulsion or liposomal nanoparticles. The liposomal nanoparticle comprises a mixture of: a lecithin; a polysorbate.

Sterile water can be included with the lecithin. The lecithin can be a complex mixture of soy derived phospholipids, glycolipids, and triglycerides having a concentration of about 0.1%-0.5% by weight of the total weight of the ophthalmic suspension base (w/w). The polysorbate can be polysorbate 80 having a concentration of about 0.1%-0.5% by weight of the total weight of the ophthalmic suspension base (w/w). The cellulose ether is hydroxypropyl methylcellulose (HPMC) having a concentration of about 0.2%-1.1% by weight of the total weight of the ophthalmic suspension base (w/w).

### 5.2 Pharmaceutical Composition

Pharmaceutical formulation in the present disclosure may be applied directly to the ocular surface, such as the front of the eye (e.g. on the cornea), under the upper eyelid, on the lower eyelid and in the cul-de-sac, etc. The topical ophthalmic composition is a liquid composition that comprises an aqueous fluid. As used herein, the term “aqueous fluid” means a fluid that is at least 75% water by weight; and in some cases, at least 90%. The liquid composition may have the form of any of the various types of liquid mixtures, such as a solution, suspension, emulsion, gel, sol, liquid foam, etc.

In some embodiments, the ophthalmic compositions may further comprise other ingredients, such as surfactants, adjuvants, buffers, antioxidants, tonicity adjusters, preservatives (e.g. EDTA, benzalkonium chloride), sodium chlorite, sodium perborate, polyquaterium-1), thickeners, or viscosity modifiers. Examples of thickeners or viscosity modifiers that could be used include carboxymethyl cellulose, hydroxymethyl cellulose, polyvinyl alcohol, polyethylene glycol, glycol 400, propylene glycol hydroxymethyl cellulose, hyaluronic acid, hydroxypropyl methylcellulose, and the like.

The pH of the ophthalmic composition may be in any suitable range, such as pH 5.0 to 8.5. The pH of the ophthalmic fluid composition may be adjusted by adding any physiologically and ophthalmically acceptable pH-adjusting acids, bases, or buffers to a suitable range. The ophthalmic compositions of the invention can further contain pharmaceutical excipients suitable for the preparation of ophthalmic formulations. Examples of such excipients are preserving agents, buffering agents, chelating agents, antioxidant agents and salts for regulating the osmotic pressure. In certain embodiments, the pH of the ophthalmic composition can be adjusted to around 6 to 8.

In certain embodiments, the ophthalmic composition is an injectable sustained-release formulation. In certain embodiments, the composition can be sustained-release in vivo in one week, two weeks, one to three months, six months or more than six months.

In another embodiment, the ophthalmic composition includes a pharmaceutically active agent. The pharmaceutically active agent can be an antibiotic, a nonsteroidal anti-inflammatory agent or any other desired agent. The pharmaceutically active agent has a concentration of about 0.1%-10.0% by weight of the total weight of the ophthalmic composition (w/w).

### 5.3 Methods of Making the Drug Delivery System

The disclosure relates to a method of making a drug delivery system comprising a nanoparticle. The nanoparticle comprising a core comprising one or more therapeutic agents and a carrier; and an outer surface surrounding the core, the outer surface comprising a membrane derived from an ocular tumor or ocular cancer.

In one embodiment, the method of making the drug delivery system comprises the steps of: (i) extracting an ocular tumor cell membrane to form an extract; (ii) loading a nanoparticle with one or more therapeutic agent and a carrier to form a core; (iii) combining the extract and the core to form the drug delivery system.

The disclosure relates to a method of preparing an ophthalmic formulation, including a method of preparing any of the ophthalmic formulations described herein. In one embodiment, provided is a method of preparing an ophthalmic formulation comprising optionally forming nanoparticles or microparticles of a therapeutic agent, a pharmaceutically acceptable salts thereof, and at least one pharmaceutically acceptable carrier.’

In one embodiment, the nanoparticles or microparticles can be formed in the presence of surfactant. Any of the surfactants described herein can be used including, but not limited to, Tween 80, Tween 20, poloxamer 188, poloxamer 407, or tyloxapol, and preferably tyloxapol. Any of the methods described herein can be used to form the nanoparticles or microparticles. In certain embodiments, milling is used, such as a ball-milling process.

Also provided is a method for preparing an ophthalmic composition comprising a delivery system and a pharmaceutically acceptable excipient, wherein the delivery system is selected from the group consisting of microspheres, microcapsules, microparticles, liposomes, multivesicular liposomes, nanocrystals and nanoparticles. The method comprises preparing the microspheres via emulsion solvent evaporation, double emulsion solvent evaporation, emulsification-chemical cross-linking method, emulsion-direct heat cross-linking method, spray drying, phase separation, SCF (supercritical fluid), ultrasonic atomization, electro spraying, hot melt extrusion, or polymer-alloys method.

In yet another aspect, the disclosure relates to a method for preparing an ophthalmic injectable sustained-release formulation comprising a delivery system and a pharmaceutically acceptable excipient, wherein the delivery system is selected from the group consisting of microspheres, microcapsules, microparticles, liposomes, multivesicular liposomes, nanocrystals and nanoparticles; and wherein the method comprises preparing the liposomes via thin-film hydration, sonication, membrane extrusion, REV (reverse phase evaporation), ether injection, ethanol injection, freezing thawing, French press method, multiple emulsion, SRPE (supercritical fluid reverse phase evaporation), freeze drying of double emulsions, or ion gradient.

In still another aspect, the disclosure relates to a method for preparing an ophthalmic injectable sustained-release formulation comprising a delivery system and a pharmaceutically acceptable excipient, wherein the delivery system is selected from the group consisting of microspheres, microcapsules, microparticles, liposomes, multivesicular liposomes, nanocrystals and nanoparticles; and wherein the method comprises preparing the multivesicular liposomes via double emulsion solvent evaporation.

### 5.4 Kits

The ophthalmic composition may be packaged in any suitable manner, such as a ready-to-use single-use product or a multi-use product. The single-use product is intended to be consumed in a single setting. The ophthalmic composition may be provided in any suitable type of eye-dropper container for topical ocular administration. In some embodiments, the product is a multi-use product and the eye-dropper container contains 3-20 mL volume of the ophthalmic composition. In one embodiment, the ophthalmic composition is packaged in a multi-use (multi-dose) eye-dropper vial containing the ophthalmic composition. In some embodiments, the product is a single-use product and the eye-dropper container contains less than 1 mL volume of the ophthalmic composition.

### 5.5 Method of Treatment or Prevention

The ophthalmic composition of the disclosure may be used for treating an eye disorder. As used herein, the term “treating” also encompasses preventing an eye disorder. One or more particular symptoms of eye disorder could be ameliorated, including eye discomfort, visual disturbance, tear film instability, tear hyperosmolarity, and inflammation of the ocular surface.

The subject receives the ophthalmic composition by injecting the composition directly onto the eye for one or both eyes. The subject receives the ophthalmic composition topically by instilling droplets directly onto the eye surface (which encompasses the conjunctival sac), for one or both eyes. Topical administration of the ophthalmic composition may be performed by the patient themselves. Or it could be performed by someone else, such as a caregiver, a spouse, a clinician (e.g. physician or nurse), etc.

The subject may receive intermittent dosing of the ophthalmic composition. In some embodiments, the ophthalmic composition is administered once daily. In some embodiments, the ophthalmic composition is administered twice daily. In some embodiments, the ophthalmic composition is administered three times daily.

### 6. EXAMPLES

Provided in this disclosure is an intraocular drug delivery for treatment of eye diseases like retinoblastoma. It takes advantage of non-systemic side effects and relatively high delivery efficiency for bypassing physiological barriers like blood-retina barrier (BRB). However, conventional intraocular delivery still has drawbacks like toxicity on off-target tissues. Provided herein is a biomimetic intraocular drug delivery system that can target retinoblastoma cells. The method is based on cancer cell membrane coating strategy. In one embodiment, membrane of human retinoblastoma cells (WERI-Rb1, RB1) is extracted and coated on drug-loaded PLGA nanoparticles. The retinal structure is maintained by intercellular binding of retinal cells dominated by retinoschisin expressed on their membrane. The RB1-coated PLGA@Drug nanoparticles targeted the RB1 cells in the eye after intravitreal injection. The RB1-coated PLGA nanoparticle was prepared and loaded it with fluorescent dye DiD. The coating was successful and the RB1-coated nanoparticles displayed significant targeting effect on the RB1 cells. Loaded with classical chemotherapy drug etoposide, the RB1-coated nanoparticles also displayed enhanced cytotoxicity on WERI-Rb1 cells. Provided herein is a first example of biomimetic intraocular drug delivery system based on cancer cell membrane coating technology for simple and efficient treatment of intraocular tumors with practical biomedical applications.

In one example, a retinoblastoma cell membrane coated drug delivery system for treatment of retinoblastoma was prepared. First, the membrane of WERI-Rb1, a human retinoblastoma cell line was extracted with commercially available extraction kits. Second, the drug loaded PLGA nanoparticles were fabricated by single emulsion method. Bare PLGA nanoparticles, dye-loaded PLGA nanoparticles and control group mPEG-PLGA nanoparticles were fabricated in the same way.

In one example, the drug etoposide was chosen as a typical anti-tumour drug. The nanoparticles were coated through ultrasound bath immersion. The WERI-Rb1 coated PLGA nanoparticles were expected to have significant targeting effect on human retinoblastoma cells (FIGS. 1A-1B). An optimization of weight ratio of membrane over drug loaded PLGA nanoparticles cores was done. Some characterizations were performed to demonstrate the successful coating. The DiD dye was encapsulated into PLGA nanoparticles to test the RB1-PLGA@DiD NPs' targeting effect on WERI-Rb1 cells.

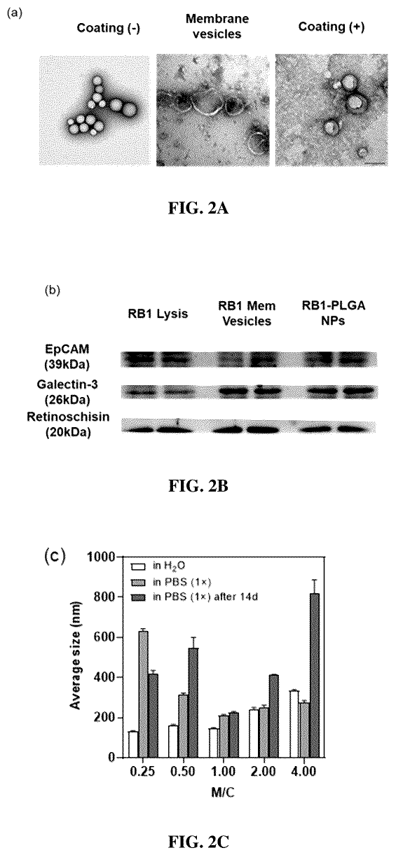

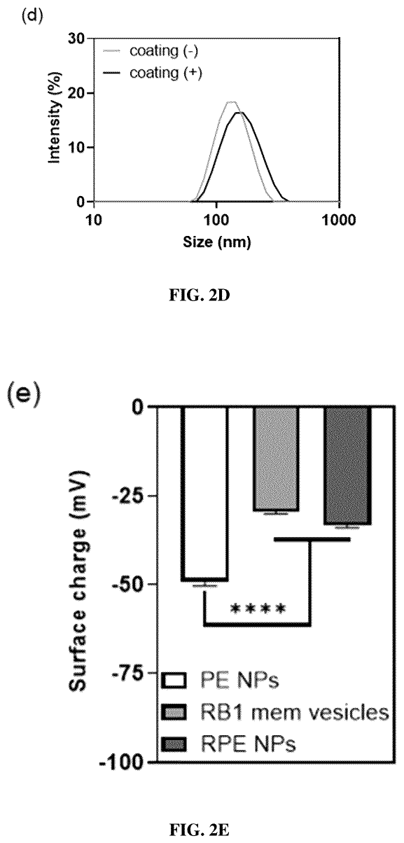

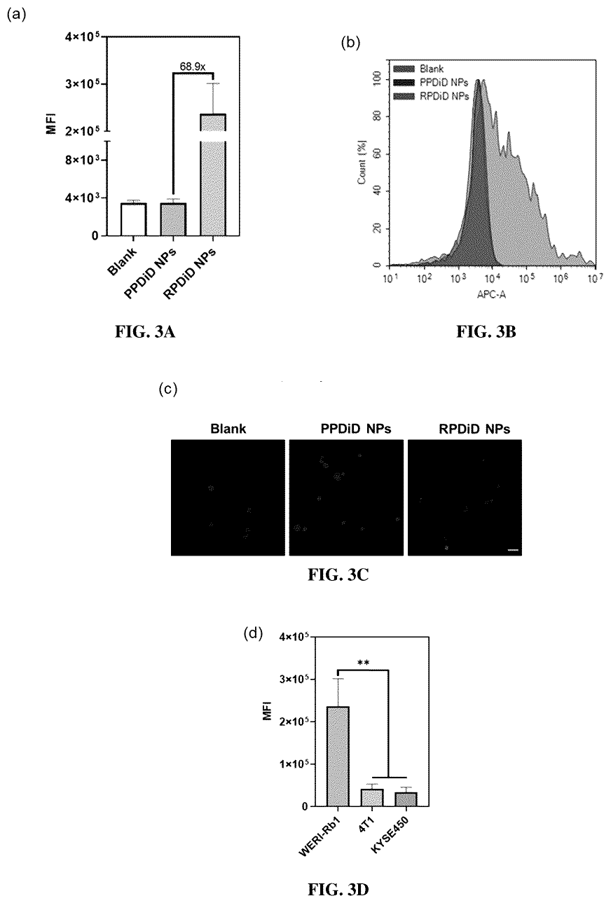

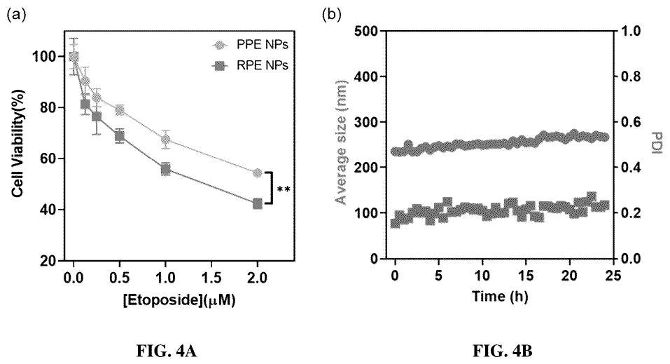

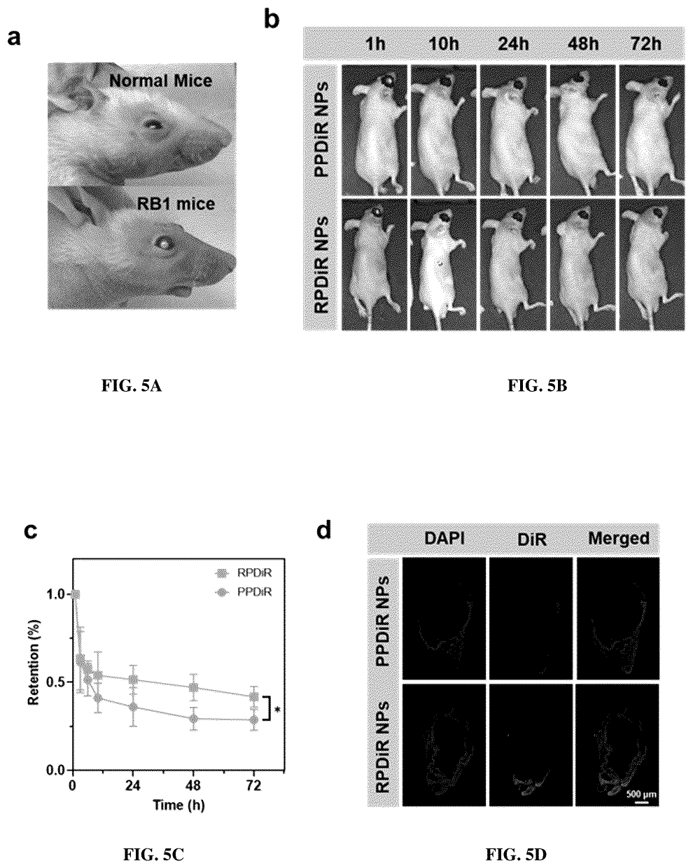

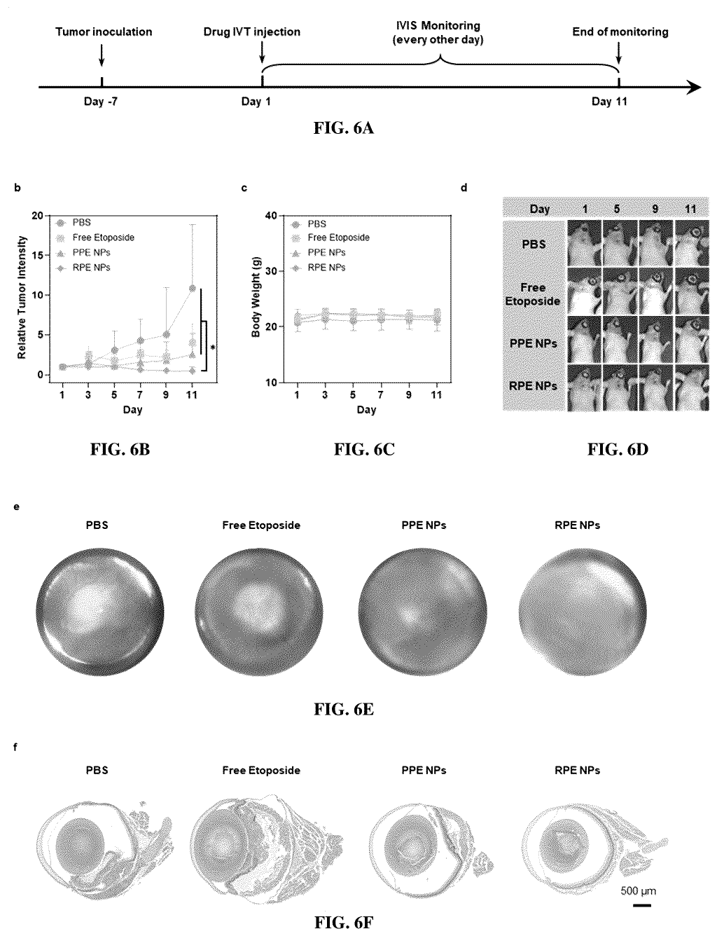

To demonstrate feasibility of RB1 membrane coating on PLGA based nanoparticles, morphology and structure of bare PLGA NPs, WERI-Rb1 membrane vesicles, and coated nanoparticles RB1-PLGA NPs were characterized by using transmission electron microscope (TEM). Image of the RB1-PLGA NPs displayed significant core-shell structure, which indicated the successful coating (FIG. 2A). Protein analysis result of RB1 cell lysis, RB1 membrane vesicles, and RB1-PLGA NPs showed that the surface proteins which dominate intercellular binding were well preserved in the coating process (FIG. 2B), which demonstrated the successful functionalization of the PLGA NPs.

General coating characterizations of drug-loaded PLGA nanoparticles were presented. By using dynamic light scattering (DLS) machine, weight ratio of membrane over PE NPs (M/C) was optimized through monitoring the RPE NPs' average size change in water, PBS buffer (1×), and PBS buffer (1×) after 14 days. As is shown (FIG. 2C), under circumstance of fixed final weight concentration of PE NPs (1 mg/mL), the ratio M/C at 1/1 displayed the best stability of formed RPE NPs. The average size change of PE NPs was tested before and after coating. According to the result, the average size of PE NPs increased from 131.2 nm to 147.8 nm after coated with WERI-Rb1 cell membrane while polydispersity index (PDI) remained to be smaller than 0.2 (FIG. 2D). Moreover, the surface charge of PE NPs, WERI-Rb1 membrane vesicles (RB1 mem vesicles), and coated nanoparticles (RPE NPs) were tested with DLS machine. The result showed that the surface charge of the nanoparticles increased to be close to level of the membrane vesicles, which was also consistent with the core-shell structure of the coated nanoparticles (FIG. 2E).

Targeting effect of the RB1-PLGA NPs on retinoblastoma cells was tested. PLGA nanoparticles were loaded with fluorescent dye DiD. With flow cytometry and confocal imaging, it is indicated that WERI-Rb1 cells have significantly higher cellular uptake of RB1-PLGA@DiD nanoparticles (RPDiD NPs) compared with control group mPEG-PLGA@DiD nanoparticles (PPDiD NPs) (FIGS. 3A, 3B and 3C). The advantage of the uptake was around 68.9 folds. Furthermore, cellular uptake of RPDiD nanoparticles on various kinds of cells was compared (FIG. 3D): human retinoblastoma cells (WERI-Rb1), mouse mammary carcinoma cells (4T1), human oesophageal squamous carcinoma cells (KYSE450). The result showed that WERI-Rb1 cell had highest uptake compared with other kinds of cells and the advantage were all significant. The targeting effect was verified consequently.

The cytotoxicity of RPE NPs on WERI-Rb1 cells was tested. As expected, RPE NPs had enhanced cytotoxicity on WERI-Rb1 cells for its targeting effect (FIG. 4A). Moreover, stability of the RPE NPs in PBS buffer (1×) in 25° C. was tested and the result showed that size and PDI of RPE NPs remained to be stable within 24 hours (FIG. 4B), which indicated its clinical translation potential. Overall, the developed intraocular drug delivery strategy provides a simple but efficient method for treatment of intraocular tumours like retinoblastoma.

Intraocular targeting and retention of the WERI-Rb-1 cell membrane-coated nanoparticles was evaluated in an orthotopic retinoblastoma-bearing mouse model. For the model construction, 2×10⁴ WERI-Rb-1-Luc-GFP cells dispersed in 2 μL PBS buffer was intravitreally injected into the bottom of the vitreous cavity of the right eye of a Balb/c nude mouse. After 7 days, leukocoria can be observed in successfully inoculated mouse (FIG. 5A). Detailed progress of the inoculated retinoblastoma was monitored by detection of its luminescence by in vivo imaging system (IVIS) with intraperitoneal injection of 150 mg/kg D-luciferin potassium salt. RB1-PLGA@DiR nanoparticles (RPDiR NPs) or mPEG-PLGA@DiR nanoparticles (PPDiR NPs) were intravitreally injected. Fluorescence signal of DiR was detected by IVIS at 3, 6, 10, 24, 48, 72 h post-injection (FIGS. 5B and 5C). As shown, RPDiR NPs presented significantly higher intraocular retention. After 72 h, the mice were euthanized, and the tumour-inoculated eyes were harvested for cryosection (FIG. 5D). DAPI-stained live cells presented longitudinal profile of the mouse eye. It could be found that DiR far-red fluorescence presented to be stronger in RPDiR NPs groups and mainly appeared at posterior segment which was the location of retinoblastoma.

For evaluation of in vivo anti-tumour efficacy, Balb/c nude mice successfully inoculated with orthotopic retinoblastoma were randomly divided into four groups according to treatments: PBS, Free etoposide, PPE NPs and RPE NPs. The formulation (2 μL) was slowly intravitreally injected with dosage of etoposide at 0.0269 mg/kg. With intraperitoneal injection of 150 mg/kg D-luciferin potassium salt, progress of retinoblastoma was monitored by detection of its luminescence by IVIS every other day within 11 days after drug administration (FIG. 6A). RPE NPs showed significantly more inhibition on tumour growth than other three groups (FIGS. 6B and 6D). Average body weight of the mice in each group was recorded (FIG. 6C). The stable body weight indicated biosafety of the RPE NPs. At 11th day, tumour-bearing eyes of the mice were taken for MICRON IV imaging (FIG. 6E). It could be observed that leukocoria in RPE NPs group disappeared. After euthanasia, the eyes were harvested for haematoxylin and eosin staining (H&E staining, FIG. 6F). It could be observed that the RPE NPs-treated eye presented elimination of tumour, which demonstrated its excellent antitumour efficacy in vivo.

Exemplary products, systems and methods are set out in the following items:

1. An ophthalmic composition suitable for intraocular administration, the composition comprising a nanoparticle, the nanoparticle comprising:
(a) a core comprising one or more therapeutic agents and a carrier; and
(b) an outer surface surrounding the core, the outer surface comprising a membrane derived from an ocular tumor or ocular cancer.

2. The ophthalmic composition of item 1, wherein the membrane is derived from a tissue associated with an ocular disease or condition of an eye.

3. The ophthalmic composition of any one of the preceding items, wherein the membrane is derived from retinoblastoma.

4. The ophthalmic composition of any one of the preceding items, wherein the membrane comprises retinoschisin.

5. The ophthalmic composition of any one of the preceding items, wherein the ocular disease or condition is selected from the group consisting of ocular cancer, ocular tumor, macular degeneration, age-related macular degeneration (AMD), glaucoma, macular edema, uveitis, diabetic retinopathy, aphakia, blepharospasm, cataract, conjunctivitis, corneal disease, dry eye syndrome; an eyelid disease, a lacrimal disease, lacrimal duct obstruction, myopia, presbyopia, a pupil disorder, corneal neovascularization, a refractive disorder, strabismus, acute macular neuroretinopathy, Behcet's disease; choroidal neovascularization, histoplasmosis, multifocal choroiditis, proliferative vitreoretinopathy (PVR), retinal arterial occlusive disease, retinal detachment, sympathetic ophthalmia, Vogt Koyanagi-Harada (VKH) syndrome, a posterior ocular condition associated with an ocular laser treatment, a posterior ocular condition associated with photodynamic therapy, photocoagulation, radiation retinopathy, epiretinal membrane disorder, branch retinal vein occlusion, anterior ischemic optic neuropathy, non-retinopathy diabetic retinal dysfunction, and retinitis pigmentosa.

6. The ophthalmic composition of any one of the preceding items, wherein the membrane is or is derived from an ocular tumor or an ocular cancer.

7. The ophthalmic composition of any one of the preceding items, wherein the membrane is derived from an ocular cancer cell or tumor selected from the group consisting of retinoblastoma, medulloepithelioma, intraocular melanoma, iris melanoma, ciliary body melanoma, choroidal melanoma, uveal melanoma, eyelid cancer, intraocular lymphoma, orbital lymphoma, and a cancer that has metastasized to the eye.

8. The ophthalmic composition of any one of the preceding items, wherein the therapeutic agent is a small molecule, a biomolecule, an enzyme, a protein, a polypeptide or a nucleic acid.

9. The ophthalmic composition of any one of the preceding items, wherein the therapeutic agent is an ophthalmic drug used to treat, prevent or diagnose an ocular disease, and wherein the therapeutic agent is selected from the group consisting of a chemotherapeutic agent, an anti-glaucoma agent, an anti-angiogenesis agent, an anti-infective agent, an anti-inflammatory agent, a growth factor, an immunosuppressant agent, an anti-allergic agent, and combinations thereof.

10. The ophthalmic composition of any one of the preceding items, wherein the therapeutic agent is a chemotherapeutic agent effective to treat an ocular cancer or an ocular tumor.

11. The ophthalmic composition of any one of the preceding items, wherein the chemotherapeutic agent is selected from the group consisting of etoposide, toposide, tebentafusp, mitomycin C, fluorouracil (5FU), methotrexate, cytarabine (Ara-C), thiotepa, chlorambucil, dacarbazine, temozolomide, teniposide, verapamil, vincristine, calcitriol, melphalan, cyclosporin, carboplatin, cisplatin, topotecan and Nutlin-3.

12. The ophthalmic composition of any one of the preceding items, wherein the carrier comprises a polymer, a lipid, a phospholipid, a dendrimer or a metal nanoparticle.

13. The ophthalmic composition of any one of the preceding items, wherein the carrier in the nanoparticle core comprises a biodegradable or biocompatible polymer.

14. The ophthalmic composition of any one of the preceding items, wherein the biodegradable or biocompatible polymer is selected from the group consisting of poly(D,L-lactide-co-glycolide) (PLGA), poly(L-lactide-co-glycolide), poly(D-lactide-co-glycolide), poly-(D-lactic acid) (PDLA), poly-(L-lactic acid) (PLLA), poly(D,L-lactic acid) (PLA), poly(glycolic acid) (PGA), poly(L-lactide-co-caprolactone), poly(D-lactide-co-caprolactone), poly(D,L-lactide-co-caprolactone) (PCL), polyvinyl alcohol (PVA), poly(ethylene glycol) (PEG), polylysine, poly(L-lactide-co-trimethylene carbonate), poly(D-lactide-co-trimethylene carbonate), poly(D,L-lactide-co-trimethylene carbonate), poly(trimethylene carbonate-co-&-caprolactone), poly(caprolactone-co-glycolide), polystyrene, polyurethane, silicone, hydroxypropylmethylcellulose (HPMC), hydroxypropylcellulose (HPC), polyglycolic acid-polyvinyl alcohol copolymers, polycaprolactone-polyethylene glycol copolymers, collagen, croscarmellose collagen, hyaluronic acid, elastin, polyhydroxybutyrate, polyalkaneanhydrides, gelatin, cellulose, oxidized cellulose, polyphosphazene, poly(sebacic acid), poly(ricenolic acid), poly(fumaric acid), chitin, chitosan, polyvinylpyrrolidone (PVP), synthetic cellulose esters, polyacrylic acids, polybutyric acid; triblock copolymers (e.g., PLGA-PEG-PLGA, PEG-PLGA-PEG), poly(N-isopropylacrylamide, poly(ethylene oxide)-poly(propylene oxide)-poly(ethylene oxide) tri-block copolymers (PEO-PPO-PEO), poly valeric acid, poly(valerolactone), polyhydroxyalkylcellulose; siloxane, polysiloxane; dimethylsiloxane/-methylvinylsiloxane copolymer; poly(dimethylsiloxane/-methylvinylsiloxane/-methylhydrogensiloxane) dimethylvinyl or trimethyl copolymer, polypeptides, poly(amino acids), poly(dioxanones), poly(alkylene alkylates), hydrophobic polyethers, polyetheresters, polyacetals, polycyanoacrylates, polyacrylates, polymethylmethacrylates, polysiloxanes, poly(oxyethylene)/poly(oxypropylene) copolymers, polyketals, polyphosphates, polyhydroxyvalerates, polyalkylene oxalates, polyalkylene succinates, poly(maleic acid), as well as copolymers thereof or derivatives thereof including oxidized, reduced, mono substituted, disubstituted, multi-substituted derivatives, or any combinations thereof.

15. The ophthalmic composition of any one of the preceding items, wherein the carrier in the nanoparticle core comprises a biodegradable polymer selected from the group consisting of poly(D,L-lactide-co-glycolide) (PLGA), poly(L-lactide-co-glycolide), poly(D-lactide-co-glycolide), poly-(D-lactic acid) (PDLA), poly-(L-lactic acid) (PLLA), poly(D,L-lactic acid) (PLA), and poly(glycolic acid) (PGA).

16. The ophthalmic composition of any one of the preceding items, wherein the weight ratio of the nanoparticle core to the membrane is about 10:1 to about 1:10.

17. The ophthalmic composition of any one of the preceding items, wherein the weight ratio of the nanoparticle core to the membrane is about 1:1.

18. The ophthalmic composition of any one of the preceding items, wherein the composition is in a form suitable for subcutaneous, subconjunctival, sub-tenon, intracameral, intravitreal or retrobulbar administration into an eye of a subject.

19. The ophthalmic composition of any one of the preceding items, wherein the ophthalmic composition is formulated as an intraocular injection or an ophthalmic implant.

20. The composition of any one of the preceding items, wherein the one or more therapeutic agents is selected from the group consisting of a chemotherapeutic agent, an anti-glaucoma agent, an anti-angiogenesis agent, an anti-infective agent, an anti-inflammatory agent, a growth factor, an immunosuppressant agent, an anti-allergic agent, and combinations thereof.

21. A method of treating an ocular disease or condition of an eye, comprising the step of intraocularly administering to a subject in need thereof an ophthalmic composition comprising a nanoparticle, the nanoparticle comprising:
(a) a core comprising one or more therapeutic agents and a carrier; and
(b) an outer surface surrounding the core, the outer surface comprising a membrane derived from an ocular tumor or ocular cancer.

22. The method of any one of the preceding items, wherein the membrane is derived from a tissue associated with an ocular disease or condition.

23. The method of any one of the preceding items, wherein the ocular disease or condition is selected from the group consisting of ocular cancer, ocular tumor, macular degeneration, age-related macular degeneration (AMD), glaucoma, macular edema, uveitis, diabetic retinopathy, aphakia, blepharospasm, cataract, conjunctivitis, corneal disease, dry eye syndrome; an eyelid disease, a lacrimal disease, lacrimal duct obstruction, myopia, presbyopia, a pupil disorder, corneal neovascularization, a refractive disorder, strabismus, acute macular neuroretinopathy, Behcet's disease; choroidal neovascularization, histoplasmosis, multifocal choroiditis, proliferative vitreoretinopathy (PVR), retinal arterial occlusive disease, retinal detachment, sympathetic ophthalmia, Vogt Koyanagi-Harada (VKH) syndrome, a posterior ocular condition associated with an ocular laser treatment, a posterior ocular condition associated with photodynamic therapy, photocoagulation, radiation retinopathy, epiretinal membrane disorder, branch retinal vein occlusion, anterior ischemic optic neuropathy, non-retinopathy diabetic retinal dysfunction, and retinitis pigmentosa.

24. The method of any one of items 21-23, wherein the membrane is derived from retinoblastoma.

25. The method of any one of the preceding items, wherein uptake of the nanoparticle into an ocular cell is increased relative to a composition lacking the membrane.

26. The method of any one of the preceding items, wherein the membrane targets the ophthalmic composition to an ocular cell of the subject.

27. The method of any one of the preceding items, wherein the membrane is or is derived from an ocular tumor or an ocular cancer.

28. The method of any one of the preceding items, wherein the membrane is derived from an ocular cancer cell selected from the group consisting of retinoblastoma, medulloepithelioma, ocular melanoma, intraocular melanoma, iris melanoma, ciliary body melanoma, choroidal melanoma, uveal melanoma, eyelid cancer, ocular lymphoma, intraocular lymphoma, orbital lymphoma, or a cancer that has metastasized to the eye.

29. The method of any one of the preceding items, wherein the membrane comprises retinoschisin.

30. The method of any one of the preceding items, wherein the therapeutic agent is a small molecule, a biomolecule, an enzyme, a protein, a polypeptide or a nucleic acid.

31. The method of any one of the preceding items, wherein the therapeutic agent is an ophthalmic drug used to treat, prevent or diagnose an ocular disease, and wherein the therapeutic agent is selected from the group consisting of a chemotherapeutic agent, an anti-glaucoma agent, an anti-angiogenesis agent, an anti-infective agent, an anti-inflammatory agent, a growth factor, an immunosuppressant agent, an anti-allergic agent, and combinations thereof.

32. The method of any one of the preceding items, wherein the therapeutic agent is a chemotherapeutic agent effective to treat an ocular cancer or an ocular tumor.

33. The method of any one of the preceding items, wherein the chemotherapeutic agent is selected from the group consisting of etoposide, toposide, tebentafusp, mitomycin C, fluorouracil (5FU), methotrexate, cytarabine (Ara-C), thiotepa, chlorambucil, dacarbazine, temozolomide, teniposide, verapamil, vincristine, calcitriol, melphalan, cyclosporin, carboplatin, cisplatin, topotecan and Nutlin-3.

34. The method of any one of the preceding items, wherein the nanoparticle core comprises a biodegradable or biocompatible polymer.

35. The method of any one of the preceding items, wherein the biodegradable or biocompatible polymer is selected from the group consisting of poly(D,L-lactide-co-glycolide) (PLGA), poly(L-lactide-co-glycolide), poly(D-lactide-co-glycolide), poly-(D-lactic acid) (PDLA), poly-(L-lactic acid) (PLLA), poly(D,L-lactic acid) (PLA), poly(glycolic acid) (PGA), poly(L-lactide-co-caprolactone), poly(D-lactide-co-caprolactone), poly(D,L-lactide-co-caprolactone) (PCL), polyvinyl alcohol (PVA), poly(ethylene glycol) (PEG), polylysine, poly(L-lactide-co-trimethylene carbonate), poly(D-lactide-co-trimethylene carbonate), poly(D,L-lactide-co-trimethylene carbonate), poly(trimethylene carbonate-co-ε-caprolactone), poly(caprolactone-co-glycolide), polystyrene, polyurethane, silicone, hydroxypropylmethylcellulose (HPMC), hydroxypropylcellulose (HPC), polyglycolic acid-polyvinyl alcohol copolymers, polycaprolactone-polyethylene glycol copolymers, collagen, croscarmellose collagen, hyaluronic acid, elastin, polyhydroxybutyrate, polyalkaneanhydrides, gelatin, cellulose, oxidized cellulose, polyphosphazene, poly(sebacic acid), poly(ricenolic acid), poly(fumaric acid), chitin, chitosan, polyvinylpyrrolidone (PVP), synthetic cellulose esters, polyacrylic acids, polybutyric acid; triblock copolymers (e.g., PLGA-PEG-PLGA, PEG-PLGA-PEG), poly(N-isopropylacrylamide, poly(ethylene oxide)-poly(propylene oxide)-poly(ethylene oxide) tri-block copolymers (PEO-PPO-PEO), poly valeric acid, poly(valerolactone), polyhydroxyalkylcellulose; siloxane, polysiloxane; dimethylsiloxane/-methylvinylsiloxane copolymer; poly(dimethylsiloxane/-methylvinylsiloxane/-methylhydrogensiloxane) dimethylvinyl or trimethyl copolymer, polypeptides, poly(amino acids), poly(dioxanones), poly(alkylene alkylates), hydrophobic polyethers, polyetheresters, polyacetals, polycyanoacrylates, polyacrylates, polymethylmethacrylates, polysiloxanes, poly(oxyethylene)/poly(oxypropylene) copolymers, polyketals, polyphosphates, polyhydroxyvalerates, polyalkylene oxalates, polyalkylene succinates, poly(maleic acid), as well as copolymers thereof or derivatives thereof including oxidized, reduced, mono substituted, disubstituted, multi-substituted derivatives, or any combinations thereof.

36. The method of any one of the preceding items, wherein the nanoparticle core comprises a biodegradable polymer selected from the group consisting of poly(D,L-lactide-co-glycolide) (PLGA), poly(L-lactide-co-glycolide), poly(D-lactide-co-glycolide), poly-(D-lactic acid) (PDLA), poly-(L-lactic acid) (PLLA), poly(D,L-lactic acid) (PLA), and poly(glycolic acid) (PGA).

37. The method of any one of the preceding items, wherein the weight ratio of the nanoparticle core to the membrane is about 10:1 to about 1:10.

38. The method of any one of the preceding items, wherein the weight ratio of the nanoparticle core to the membrane is about 1:1.

39. The method of any one of the preceding items, wherein the ophthalmic composition is in a form suitable for intraocular administration.

40. The method of any one of the preceding items, wherein the ophthalmic composition is in a form suitable for subcutaneous, subconjunctival, sub-tenon, intracameral, intravitreal or retrobulbar administration, or in a form suitable for intravitreal administration.

41. The method of any one of the preceding items, wherein the ophthalmic composition is formulated as an intraocular injection or an ophthalmic implant.

42. The method of any one of the preceding items, wherein the one or more therapeutic agents is selected from the group consisting of a chemotherapeutic agent, an anti-glaucoma agent, an anti-angiogenesis agent, an anti-infective agent, an anti-inflammatory agent, a growth factor, an immunosuppressant agent, an anti-allergic agent, and combinations thereof.

43. The method of any one of the preceding items, wherein the therapeutic agent is in a range of about 0.01-0.04 mg/kg.

44. The method of any one of the preceding items, wherein the therapeutic agent is in the range of about 0.0269 mg/kg.

45. The method of any one of the preceding items, wherein the therapeutic agent is Etoposide.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments but should be defined only in accordance with the following claims and their equivalents.

All references cited herein are incorporated herein by reference in their entirety and for all purposes to the same extent as if each individual publication or patent or patent application was specifically and individually indicated to be incorporated by reference in its entirety for all purposes.

### REFERENCES

(1) Dimaras, H.; Kimani, K.; Dimba, E. A. O.; Gronsdahl, P.; White, A.; Chan, H. S. L.; Gallie, B. L. Retinoblastoma. The Lancet 2012, 379 (9824), 1436-1446. DOI: https://doi.org/10.1016/S0140-6736 (11) 61137-9.
(2) Hosoya, K.-i.; Tachikawa, M. Inner Blood-Retinal Barrier Transporters: Role of Retinal Drug Delivery. Pharmaceutical Research 2009, 26 (9), 2055-2065. DOI: 10.1007/s11095-009-9930-2.
(3) Dimaras, H.; Corson, T. W.; Cobrinik, D.; White, A.; Zhao, J.; Munier, F. L.; Abramson, D. H.; Shields, C. L.; Chantada, G. L.; Njuguna, F.; et al. Retinoblastoma. Nature Reviews Disease Primers 2015, 1 (1), 15021. DOI: 10.1038/nrdp.2015.21.
(4) Yoo, J.-W.; Irvine, D. J.; Discher, D. E.; Mitragotri, S. Bio-inspired, bioengineered and biomimetic drug delivery carriers. Nature Reviews Drug Discovery 2011, 10 (7), 521-535. DOI: 10.1038/nrd3499.
(5) Lang, T.; Yin, Q.; Li, Y. Progress of Cell-Derived Biomimetic Drug Delivery Systems for Cancer Therapy. Advanced Therapeutics 2018, 1 (7), 1800053. DOI: https://doi.org/10.1002/adtp.201800053.
(6) Kroll, A. V.; Fang, R. H.; Zhang, L. Biointerfacing and Applications of Cell Membrane-Coated Nanoparticles. Bioconjugate Chemistry 2017, 28 (1), 23-32. DOI: 10.1021/acs.bioconjchem.6b00569.
(7) Harris, J. C.; Scully, M. A.; Day, E. S. Cancer Cell Membrane-Coated Nanoparticles for Cancer Management. Cancers 2019, 11 (12), 1836.
(8) Heymann, J. B.; Vijayasarathy, C.; Fariss, R. N.; Sieving, P. A. Advances in understanding the molecular structure of retinoschisin while questions remain of biological function. Progress in Retinal and Eye Research 2022, 101147. DOI: https://doi.org/10.1016/j.preteyeres.2022.101147.
(9) Vijayasarathy, C.; Ziccardi, L.; Sieving, P. A. Biology of Retinoschisin. Boston, MA, 2012; Springer US: pp 513-518.
(10) Grayson, C.; Reid, S. N. M.; Ellis, J. A.; Rutherford, A.; Sowden, J. C.; Yates, J. R. W.; Farber, D. B.; Trump, D. Retinoschisin, the X-linked retinoschisis protein, is a secreted photoreceptor protein, and is expressed and released by Weri-Rb1 cells. Human Molecular Genetics 2000, 9 (12), 1873-1879. DOI: 10.1093/hmg/9.12.1873 (accessed Dec. 15, 2022).
(11) Fang, R. H.; Hu, C.-M. J.; Luk, B. T.; Gao, W.; Copp, J. A.; Tai, Y.; O'Connor, D. E.; Zhang, L. Cancer Cell Membrane-Coated Nanoparticles for Anticancer Vaccination and Drug Delivery. Nano Letters 2014, 14 (4), 2181-2188. DOI: 10.1021/n1500618u.
(12) Liu, X.; Sun, Y.; Xu, S.; Gao, X.; Kong, F.; Xu, K.; Tang, B. Homotypic Cell Membrane-Cloaked Biomimetic Nanocarrier for the Targeted Chemotherapy of Hepatocellular Carcinoma. Theranostics 2019, 9 (20), 5828-5838. DOI: 10.7150/thno.34837 From NLM. Li, S.-Y.; Cheng, H.; Qiu, W.-X.; Zhang, L.; Wan, S.-S.; Zeng, J.-Y.; Zhang, X.-Z. Cancer cell membrane-coated biomimetic platform for tumor targeted photodynamic therapy and hypoxia-amplified bioreductive therapy. Biomaterials 2017, 142, 149-161. DOI: https://doi.org/10.1016/j.biomaterials.2017.07.026.
(13) Chen, L.; Wu, F.; Pang, Y.; Yan, D.; Zhang, S.; Chen, F.; Wu, N.; Gong, D.; Liu, J.; Fu, Y.; et al. Therapeutic nanocoating of ocular surface. Nano Today 2021, 41, 101309. DOI: https://doi.org/10.1016/j.nantod.2021.101309.

What is claimed:

1. An ophthalmic composition suitable for intraocular administration, the composition comprising a nanoparticle, the nanoparticle comprising:
(a) a core comprising one or more therapeutic agents and a carrier; and
(b) an outer surface surrounding the core, the outer surface comprising a membrane derived from an ocular tumor or ocular cancer.

2. The ophthalmic composition of claim 1, wherein the membrane is derived from retinoblastoma.

3. The ophthalmic composition of claim 1, wherein the membrane comprises retinoschisin.

4. The ophthalmic composition of claim 1, wherein the therapeutic agent is an ophthalmic drug used to treat, prevent or diagnose an ocular disease, and wherein the therapeutic agent is selected from the group consisting of a chemotherapeutic agent, an anti-glaucoma agent, an anti-angiogenesis agent, an anti-infective agent, an anti-inflammatory agent, a growth factor, an immunosuppressant agent, an anti-allergic agent, and combinations thereof.

5. The ophthalmic composition of claim 1, wherein the therapeutic agent is a chemotherapeutic agent effective to treat an ocular cancer or an ocular tumor.

6. The ophthalmic composition of claim 5, wherein the chemotherapeutic agent is selected from the group consisting of etoposide, toposide, tebentafusp, mitomycin C, fluorouracil (5FU), methotrexate, cytarabine (Ara-C), thiotepa, chlorambucil, dacarbazine, temozolomide, teniposide, verapamil, vincristine, calcitriol, melphalan, cyclosporin, carboplatin, cisplatin, topotecan and Nutlin-3.

7. The ophthalmic composition of claim 1, wherein the carrier comprises a polymer, a lipid, a phospholipid, a dendrimer or a metal nanoparticle.

8. The ophthalmic composition of claim 1, wherein the carrier in the nanoparticle core comprises a biodegradable or biocompatible polymer.

9. The ophthalmic composition of claim 8, wherein the biodegradable or biocompatible polymer is selected from the group consisting of poly(D,L-lactide-co-glycolide) (PLGA), poly(L-lactide-co-glycolide), poly(D-lactide-co-glycolide), poly-(D-lactic acid) (PDLA), poly-(L-lactic acid) (PLLA), poly(D,L-lactic acid) (PLA), poly(glycolic acid) (PGA), poly(L-lactide-co-caprolactone), poly(D-lactide-co-caprolactone), poly(D,L-lactide-co-caprolactone) (PCL), polyvinyl alcohol (PVA), poly(ethylene glycol) (PEG), polylysine, poly(L-lactide-co-trimethylene carbonate), poly(D-lactide-co-trimethylene carbonate), poly(D,L-lactide-co-trimethylene carbonate), poly(trimethylene carbonate-co-ε-caprolactone), poly(caprolactone-co-glycolide), polyorthoesters, polyanhydrides, polyesters, polyamides, polyesteramides, polycarbonates, polyethylene, polypropylene, polygalactic acid, polyacrylamide, polystyrene, polyurethane, silicone, hydroxypropylmethylcellulose (HPMC), hydroxypropylcellulose (HPC), polyglycolic acid-polyvinyl alcohol copolymers, polycaprolactone-polyethylene glycol copolymers, collagen, croscarmellose collagen, hyaluronic acid, elastin, polyhydroxybutyrate, polyalkaneanhydrides, gelatin, cellulose, oxidized cellulose, polyphosphazene, poly(sebacic acid), poly(ricenolic acid), poly(fumaric acid), chitin, chitosan, polyvinylpyrrolidone (PVP), synthetic cellulose esters, polyacrylic acids, polybutyric acid; triblock copolymers (e.g., PLGA-PEG-PLGA, PEG-PLGA-PEG), poly(N-isopropylacrylamide, poly(ethylene oxide)-poly(propylene oxide)-poly(ethylene oxide) tri-block copolymers (PEO-PPO-PEO), poly valeric acid, poly(valerolactone), polyhydroxyalkylcellulose; siloxane, polysiloxane; dimethylsiloxane/-methylvinylsiloxane copolymer; poly(dimethylsiloxane/-methylvinylsiloxane/-methylhydrogensiloxane) dimethylvinyl or trimethyl copolymer, polypeptides, poly(amino acids), poly(dioxanones), poly(alkylene alkylates), hydrophobic polyethers, polyetheresters, polyacetals, polycyanoacrylates, polyacrylates, polymethylmethacrylates, polysiloxanes, poly(oxyethylene)/poly(oxypropylene) copolymers, polyketals, polyphosphates, polyhydroxyvalerates, polyalkylene oxalates, polyalkylene succinates, poly(maleic acid), as well as copolymers thereof or derivatives thereof including oxidized, reduced, mono substituted, disubstituted, multi-substituted derivatives, or any combinations thereof.

10. The ophthalmic composition of claim 1, wherein the carrier in the nanoparticle core comprises a biodegradable polymer selected from the group consisting of poly(D,L-lactide-co-glycolide) (PLGA), poly(L-lactide-co-glycolide), poly(D-lactide-co-glycolide), poly-(D-lactic acid) (PDLA), poly-(L-lactic acid) (PLLA), poly(D,L-lactic acid) (PLA), and poly(glycolic acid) (PGA).

11. The ophthalmic composition of claim 1, wherein the weight ratio of the nanoparticle core to the membrane is about 10:1 to about 1:10, or about 1:1.

12. The ophthalmic composition of claim 1, wherein the composition is in a form suitable for subcutaneous, subconjunctival, sub-tenon, intracameral, intravitreal or retrobulbar administration into an eye of a subject.

13. The ophthalmic composition of claim 1, wherein the ophthalmic composition is formulated as an intraocular injection or an ophthalmic implant.

14. A method of treating an ocular disease or condition of an eye, comprising the step of intraocularly administering to a subject in need thereof an ophthalmic composition comprising a nanoparticle, the nanoparticle comprising:
(a) a core comprising one or more therapeutic agents and a carrier; and
(b) an outer surface surrounding the core, the outer surface comprising a membrane derived from an ocular tumor or ocular cancer.

15. The method of claim 14, wherein the membrane is derived from retinoblastoma.

16. The method of claim 14, wherein uptake of the nanoparticle into an ocular cell is increased relative to a composition lacking the membrane.

17. The method of claim 14, wherein the membrane targets the ophthalmic composition to an ocular cell of the subject.

18. The method of claim 14, wherein the membrane comprises retinoschisin.

19. The method of claim 14, wherein the therapeutic agent is a small molecule, a biomolecule, an enzyme, a protein, a polypeptide or a nucleic acid.

20. The method of claim 14, wherein the therapeutic agent is an ophthalmic drug used to treat, prevent or diagnose an ocular disease, and wherein the therapeutic agent is selected from the group consisting of a chemotherapeutic agent, an anti-glaucoma agent, an anti-angiogenesis agent, an anti-infective agent, an anti-inflammatory agent, a growth factor, an immunosuppressant agent, an anti-allergic agent, and combinations thereof.

21. The method of claim 14, wherein the therapeutic agent is a chemotherapeutic agent effective to treat an ocular cancer or an ocular tumor.

22. The method of claim 21, wherein the chemotherapeutic agent is selected from the group consisting of etoposide, toposide, tebentafusp, mitomycin C, fluorouracil (5FU), methotrexate, cytarabine (Ara-C), thiotepa, chlorambucil, dacarbazine, temozolomide, teniposide, verapamil, vincristine, calcitriol, melphalan, cyclosporin, carboplatin, cisplatin, topotecan and Nutlin-3.

23. The method of claim 14, wherein the nanoparticle core comprises a biodegradable or biocompatible polymer.

24. The method of claim 14, wherein the nanoparticle core comprises a biodegradable polymer selected from the group consisting of poly(D,L-lactide-co-glycolide) (PLGA), poly(L-lactide-co-glycolide), poly(D-lactide-co-glycolide), poly-(D-lactic acid) (PDLA), poly-(L-lactic acid) (PLLA), poly(D,L-lactic acid) (PLA), and poly(glycolic acid) (PGA).

25. The method of claim 14, wherein the weight ratio of the nanoparticle core to an ocular cancer cell membrane is about 10:1 to about 1:10, or about 1:1.

26. The method of claim 14, wherein the ophthalmic composition is in a form suitable for subcutaneous, subconjunctival, sub-tenon, intracameral, intravitreal or retrobulbar administration, or in a form suitable for intravitreal administration.

27. The method of claim 14, wherein the ophthalmic composition is formulated as an intraocular injection or an ophthalmic implant.

28. The method of claim 14, wherein the therapeutic agent is in a range of about 0.01-0.04 mg/kg.

29. The method of claim 28 wherein the therapeutic agent is in the range of about 0.0269 mg/kg.

30. The method of claim 29 wherein the therapeutic agent is Etoposide.
